(19) United States
(12) Patent Application Publication
(10) Pub. No.: US 2022/0411867 A1
(43) Pub. Date: Dec. 29, 2022

(54) ASSESSMENT OF AN ANALYTE FROM A BIOLOGICAL SAMPLE DISPOSED ON A SUPPORT

(71) Applicant: PerkinElmer Genetics, Inc., Pittsburgh, PA (US)

(72) Inventors: Sara Smith, Pittsburgh, PA (US); Erica Fox, Pittsburgh, PA (US)

(21) Appl. No.: 17/359,858

(22) Filed: Jun. 28, 2021

(51) Int. Cl. C12Q 1/6876

(52) U.S. Cl. CPC C12Q 1/6876

(57) ABSTRACT

Methods of assessing an analyte in a blood sample are provided according to aspects of the present disclosure which include: extracting the analyte from a biological sample dried on a treated support, producing an extracted sample, the treated support comprising a protein denaturant, wherein the analyte is a substrate for an enzyme present, or suspected of being present, in the biological sample, wherein the protein denaturant inhibits enzymatic activity of the enzyme on the analyte; and subjecting the extracted sample to liquid chromatography tandem mass spectrometry (LC/MS/MS), thereby assessing the analyte in the biological sample.

### BACKGROUND OF THE INVENTION

Assessment of analytes in biological samples is useful in numerous applications, such as assessing physiological status of a subject, including monitoring health and/or disease states. However, lability of analytes can confound analysis methods. In particular, biological samples contain enzymes and their substrates. Continuous enzymatic activity in the biological sample after it is obtained from a subject can result in inaccurate assessment of an analyte.

Nicotinamide adenine dinucleotide (NAD) is well-known as a cofactor for various metabolic reactions. In addition, NAD is a co-substrate in several enzymatic reactions, including ADP-ribosylation by poly(ADP-ribose) polymerases (PARPs), protein deacetylation by sirtuins, and formation of cyclic ADP-ribose by ADP-ribosyl cyclase, all of which destroy NAD as part of the enzymatic reaction.

NAD levels have been correlated to susceptibility to age-related diseases such as type 2 diabetes mellitus, cardiovascular disease, inflammation, and neurodegenerative disorders. Reductions in NAD levels suggest an increased risk of disease, see Elhassan, Y.; Philp, A.; Lavery, G., Journal of the Endocrine Society, 2017, 1 (7), 816-835.

However, the lability of NAD in biological samples due to enzyme-mediated degradation has hindered development of clinical testing.

There is a continuing need for methods for assessing enzymatically labile analytes in biological samples.

### SUMMARY OF THE INVENTION

Method of assessing nicotinamide adenine dinucleotide (NAD) in a blood sample are provided according to aspects of the present disclosure which include: applying a protein denaturant to a support, producing a treated support; applying a blood sample to the treated support, the blood sample comprising or suspected of comprising NAD, wherein the NAD is a substrate for an enzyme present, or suspected of being present, in the blood sample, wherein the protein denaturant inhibits enzymatic activity of the enzyme on NAD; drying the blood sample on the treated support, producing a test sample; extracting NAD from the test sample, producing an extracted sample; and subjecting the extracted sample to liquid chromatography tandem mass spectrometry (LC/MS/MS), thereby assessing NAD in the blood sample. NAD is detectable at a concentration of 5 μM or higher in the blood sample according to aspects of the present disclosure.

Method of assessing nicotinamide adenine dinucleotide (NAD) in a blood sample are provided according to aspects of the present disclosure which include: applying a protein denaturant to a support, producing a treated support; applying a blood sample to the treated support, the blood sample comprising or suspected of comprising NAD, wherein the NAD is a substrate for an enzyme present, or suspected of being present, in the blood sample, wherein the protein denaturant inhibits enzymatic activity of the enzyme on NAD; drying the blood sample on the treated support, producing a test sample; extracting NAD from the test sample, producing an extracted sample; and subjecting the extracted sample to liquid chromatography tandem mass spectrometry (LC/MS/MS), wherein a reference is added to the blood sample, the test sample, or both the blood sample and the test sample, thereby assessing NAD in the blood sample. NAD is detectable at a concentration of 5 μM or higher in the blood sample according to aspects of the present disclosure.

Methods of assessing an analyte in a blood sample are provided according to aspects of the present disclosure which include: applying a protein denaturant to a support, producing a treated support; applying a biological sample to the treated support, the biological sample comprising or suspected of comprising an analyte, wherein the analyte is a substrate for an enzyme present, or suspected of being present, in the biological sample, wherein the protein denaturant inhibits enzymatic activity of the enzyme on the analyte; drying the biological sample on the treated support, producing a test sample; extracting the analyte from the treated support, producing an extracted sample; and subjecting the extracted sample to liquid chromatography tandem mass spectrometry (LC/MS/MS), thereby assessing the analyte in the biological sample.

Methods of assessing an analyte in a blood sample are provided according to aspects of the present disclosure which include: extracting the analyte from a biological sample dried on a treated support, producing an extracted sample, the treated support comprising a protein denaturant, wherein the analyte is a substrate for an enzyme present, or suspected of being present, in the biological sample, wherein the protein denaturant inhibits enzymatic activity of the enzyme on the analyte; and subjecting the extracted sample to liquid chromatography tandem mass spectrometry (LC/MS/MS), thereby assessing the analyte in the biological sample.

According to aspects of the present disclosure, the support is, or includes, filter paper.

According to aspects of the present disclosure, the support is substantially planar.

According to aspects of the present disclosure, the blood sample is obtained from a subject and according to further aspects of the present disclosure, the subject is human.

According to aspects of the present disclosure, the test sample is stored for a first period of time at a first storage temperature following the drying and prior to the extracting.

According to aspects of the present disclosure, the test sample is stored for a second period of time following the first period of time and prior to the extracting, at a second storage temperature which is different than the first storage temperature.

According to aspects of the present disclosure, the test sample is stored for a first period of time in the range of 1 hour to 2 years, but may be stored for more or less time following the drying and prior to the extracting.

According to aspects of the present disclosure, the test sample is stored for a second period of time in the range of 1 hour to 2 years, but may be stored for more or less time following the first period of time and prior to the extracting.

According to aspects of the present disclosure, the test sample is stored at a first storage temperature in the range of −70° C. to 40° C., but may be stored at a higher or lower storage temperature, following the drying and prior to the extracting.

According to aspects of the present disclosure, the test sample is stored at a second storage temperature which is different than the first storage temperature in the range of −70° C. to 40° C., but may be stored at a higher or lower storage temperature, following the first period and prior to the extracting.

According to aspects of the present disclosure, the extracting is performed within one hour after the drying.

According to aspects of the present disclosure, the test sample is stored at a first storage temperature in the range of 15° C. to 30° C., but may be stored at a higher or lower first storage temperature, following the drying and prior to the extracting.

According to aspects of the present disclosure, the test sample is stored at a first storage temperature in the range of −70° C. to 4° C., but may be stored at a higher or lower second storage temperature, following the drying and prior to the extracting.

According to aspects of the present disclosure, the test sample is stored at a first storage temperature in the range of −25° C. to −15° C., but may be stored at a higher or lower second storage temperature, following the drying and prior to the extracting.

According to aspects of the present disclosure, the test sample is stored at a second storage temperature in the range of −70° C. to 4° C., but may be stored at a higher or lower second storage temperature, following the first period of time and prior to the extracting.

According to aspects of the present disclosure, the test sample is stored at a second storage temperature in the range of −25° C. to −15° C., but may be stored at a higher or lower second storage temperature, following the first period of time and prior to the extracting.

According to aspects of the present disclosure, the test sample is stored at a first storage temperature in the range of 15° C. to 30° C. for a first period of time, but may be stored at a higher or lower first storage temperature, following the drying and prior to the extracting; and the test sample is stored at a second storage temperature in the range of −25° C. to −15° C. for a second period of time, but may be stored at a higher or lower second storage temperature, following the first period of time and prior to the extracting.

### BRIEF DESCRIPTION OF THE DRAWINGS

FIG. 1A is a graph demonstrating non-linear detection of NAD due to enzymatic degradation in freshly dried blood samples;

FIG. 1B is a graph demonstrating non-linear detection of NAD due to enzymatic degradation in dried blood samples stored for three days;

FIG. 2A is a graph demonstrating linear detection of NAD in freshly dried blood samples where the samples were contacted with an enzyme inhibitor prior to drying;

FIG. 2B is a graph demonstrating linear detection of NAD in dried blood samples stored for three days where the samples were contacted with an enzyme inhibitor prior to drying;

FIG. 3 is a graph showing results of assessing NAD concentrations measured from seven different supports, including 6 treated supports (Treated Supports #1, #2, #3, #4, #5, #6) and 1 untreated support; treated Supports #2, #3, #4, #5, #6 have increasing amounts of a protein denaturant; and

FIG. 4 is a graph showing percentage increase in measured NAD concentration in samples from treated Supports #2, #3, #4, #5, #6 with increasing amounts of a protein denaturant; in comparison to Treated Support #1.

### DETAILED DESCRIPTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Herdewijn, P. (Ed.), Oligonucleotide Synthesis: Methods and Applications, Methods in Molecular Biology, Humana Press, 2004; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, Pa.: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

Methods of assessing an analyte in a biological sample are provided according to aspects of the present disclosure which include: applying an effective amount of a protein denaturant to a support, producing a treated support; applying a biological sample to the treated support, the biological sample comprising or suspected of comprising the analyte, wherein the analyte is a substrate for an enzyme present, or suspected of being present, in the biological sample, wherein the protein denaturant inhibits enzymatic activity of the enzyme on the analyte; drying the biological sample on the treated support, producing a test sample; extracting the analyte from the treated support, producing an extracted sample; and subjecting the extracted sample to liquid chromatography tandem mass spectrometry (LC/MS/MS), thereby assessing the analyte in the biological sample.

An analyte assessed according to aspects of the present disclosure is a non-protein analyte degraded by enzymatic activity in a sample.

According to aspects of the present disclosure, the analyte is a non-protein substrate or co-substrate for one or more enzymes. Examples of such analytes include, but are not limited to, DNA, RNA, oligonucleotides, dinucleotides, nucleotides, carbohydrates, starches, oligosaccharides, disaccharides, monosaccharides, lipids, triglycerides, urea, and hydrogen peroxide.

According to aspects of the present disclosure, the analyte is nicotinamide adenine dinucleotide (NAD).

The term “assessing” includes any form of measurement, and includes determining if an analyte is present or not as well as both quantitative and qualitative determinations. The terms “determining,” “measuring,” and “assessing,” and “assaying” are used interchangeably and include both quantitative and qualitative determinations.

As used herein, the term “biological sample” means any biological fluid, cell, tissue, or fraction thereof, which includes or is suspected of including an analyte and which includes or is suspected of including an enzyme wherein the analyte is a substrate or co-substrate of the enzyme which is degraded by the enzyme.

The term “degraded” used herein in reference to an action of an enzyme on an analyte, wherein the analyte is a substrate or co-substrate of the enzyme, refers to alteration of the analyte by enzymatic activity effective to break one or more covalent bonds of the analyte.

A biological sample can be, but is not limited to, urine, blood, saliva, semen, sputum, cerebral spinal fluid, tears, mucus, biopsy material, or a combination of any two or more thereof.

A biological sample can be, for example, obtained from a subject or can be derived from material obtained from a subject.

The term “subject” as used herein refers to any of various mammalian and non-mammalian organisms, including, but not limited to, humans, non-human primates, rodents, rabbits, dogs, cats, horses, cattle, pigs, goats, sheep, fish and other aquatic organisms, birds, poultry, insects, reptiles, and amphibians. According to particular aspects of the present disclosure, the subject is human.

The term “protein denaturant” as used herein refers to a chemical agent that specifically or non-specifically denatures the enzyme, and thereby inhibits enzymatic activity of an enzyme on its corresponding substrate and/or co-substrate, i.e., the analyte to be assessed, such that formation of a product of the enzyme activity by activity of an enzyme on its corresponding substrate and/or co-substrate, i.e., the analyte to be assessed, is undetectable or insignificant.

The terms “denaturation,” “denatured,” and grammatical equivalents as used herein refer to a structural change in the enzyme that results in the loss of its ability to function as an enzyme. A denatured enzyme does not have its usual structural characteristics, such as quaternary structure, tertiary structure, secondary structure, or primary structure, required for activity of the enzyme. A denatured enzyme may be permanently denatured by a protein denaturant.

Activity of a protein denaturant to denature an enzyme can be assessed by any of various methods such as differential scanning calorimetry, circular dichroism spectroscopy, use of fluorescent probes to monitor the conformational state of the enzyme, pulse proteolysis, protein crystallization, and hydrogen-exchange-mass spectroscopy.

According to particular aspects of the present disclosure, the protein denaturant does not substantially interfere with liquid chromatography or mass spectrometry analysis.

Non-limiting examples of protein denaturants included in methods according to aspects of the present disclosure illustratively include, but are not limited to, acids, such as acetic acid, trichloroacetic acid, and sulfosalicylic acid; bases; cross-linkers, such as formaldehyde and glutaraldehyde; chaotropic agents, such as urea, thiourea, and guanidine salts, such as guanidinium chloride, and guanidinium thiocyanate; lithium salts such as lithium chloride, lithium bromide, lithium acetate; surfactants such as sodium dodecyl sulfate (SDS); and reducing agents, such as 2-mercaptoethanol, dithiotheitol, and tris(2-carboxyethyl)phosphine.

According to particular aspects of the present disclosure, the protein denaturant does not include ethylenediaminetetraacetic acid (EDTA).

A support included for use in a method according to aspects of the present disclosure operates to retain a protein denaturant, wherein the combination of the support and protein denaturant is referred to as a “treated support” herein. Thus, according to particular aspects of the present disclosure, the protein denaturant is applied to a support, producing a treated support. The treated support can be stored for later use in a method of assessing an analyte.

A support included for use in a method according to aspects of the present disclosure is a solid or semi-solid to which a protein denaturant can be absorbed or adsorbed. The support can be made of, or include, any of various materials such as glass; metal, and a natural or synthetic polymer. Natural or synthetic polymers that can be included in a support include, but are not limited to, polypropylene, polycarbonate, polyester, polystyrene, nylon, cellulose, agarose, dextran, polyacrylamide, silicon; nitrocellulose, and any two or more thereof. A support included for use in a method according to aspects of the present disclosure to which a protein denaturant can be absorbed or adsorbed can be made of, or include, paper, such as filter paper, and/or a fabric, such as a woven or nonwoven fabric, or any combination thereof. A support included for use in a method according to aspects of the present disclosure to which a protein denaturant can be absorbed or adsorbed can be made of, or include, cellulose filter paper, cellulose non-woven fabric, glass fiber filter paper, glass fiber non-woven fabric, polyethylene filter paper, polyethylene non-woven fabric, polyester filter paper, polyester non-woven fabric, or any combination thereof.

A support included for use in a method according to aspects of the present disclosure to which a protein denaturant can be absorbed or adsorbed can be made of, or include, cotton linter filter paper. In certain aspects, the support can be made of, or include, cotton linter filter paper grade Ahlstrom 226. In certain aspects, the support is a dried blood spot card, and in some embodiments, is the PerkinElmer 226 sample collection device produced by PerkinElmer, Inc.

The support can be in any of various forms or shapes, including substantially planar, such as sheets, strips, cards, chips, slides, and plates.

According to aspects of the present disclosure, a support includes a characteristic or pattern detectable to provide information. For example, the support may be encoded using an optical, chemical, physical, or electronic tag. According to aspects of the present disclosure, a support includes a detectable characteristic or pattern, e.g., patient information, and/or a location for application of a biological sample, i.e., a region where the protein denaturant is present on the support.

The protein denaturant is applied to the support, producing a treated support. The protein denaturant may be applied by methods including, but not limited to, spotting, dipping, spraying, painting, printing, and stamping. The protein denaturant may cover all, or nearly all of the support or may be applied in a limited area or areas of the support.

The treated support may be used immediately or may be used at a later time. The treated support may be stored prior to use.

Following application of the protein denaturant, a biological sample is applied to the treated support such that the biological sample contacts the protein denaturant, producing a test sample.

According to aspects of the present disclosure, the biological sample is dried on the treated support, producing a test sample.

Drying the biological sample on the treated support may be performed at a temperature in the range of −70° C. to 95° C., such as −20° C. to 40° C., such as 4° C. to 37° C., such as 15° C. to 30° C. According to aspects of the present disclosure, drying the biological sample on the treated support includes exposure to an evaporative environment at a temperature in the range of −70° C. to 95° C., such as −20° C. to 40° C., such as 4° C. to 37° C., or such as 15° C. to 30° C., for desorption of water or other liquid in the biological sample on the treated support.

Drying the biological sample may, but does not necessarily, remove all water, or other liquid, from the biological sample on the treated support. Thus, drying the biological sample may remove 10%, 20%, 30%, 40%, 50%, 60%, 70%, 80%, 90%, 95%, 96%, 97%, 98%, 99%, or more, of the water, or other liquid, from the biological sample on the treated support.

According to aspects of the present disclosure, the test sample is stored at a first storage temperature for a first period of time following the drying and prior to the extracting. The first storage temperature is typically in the range of −70° C. to 40° C., such as −20° C. to 37° C., such as 4° C. to 35° C., or such as 15° C. to 30° C.

According to aspects of the present disclosure, the test sample is stored at a first storage temperature in the range of −20° C. to 4° C. According to aspects of the present disclosure, the first storage temperature is in the range of −25° C. to −15° C. According to aspects of the present disclosure, the first storage temperature is −25° C., −24° C., −23° C., −22° C., −21° C., −20° C., −19° C., −18° C., −17° C., −16° C., or −15° C.

According to aspects of the present disclosure, the test sample is stored at a first storage temperature of 15° C., 16° C., 17° C., 18° C., 19° C., 20° C., 21° C., 22° C., 23° C., 24° C., 25° C., 26° C., 27° C., 28° C., 29° C., or 30° C.

According to aspects of the present disclosure, the test sample is stored at a second storage temperature for a second period of time following the first period of time and prior to the extracting, wherein the first storage temperature is different than the second storage temperature.

The second storage temperature is typically in the range of −70° C. to 40° C., such as −20° C. to 37° C., such as 4° C. to 35° C., or such as 15° C. to 30° C.

According to aspects of the present disclosure, the test sample is stored at a second storage temperature in the range of −20° C. to 4° C. According to aspects of the present disclosure, the second storage temperature is in the range of −25° C. to −15° C. According to aspects of the present disclosure, the second storage temperature is in the range of −25° C., −24° C., −23° C., −22° C., −21° C., −20° C., −19° C., −18° C., −17° C., −16° C., or −15° C.

According to aspects of the present disclosure, the second storage temperature is in the range of 15° C., 16° C., 17° C., 18° C., 19° C., 20° C., 21° C., 22° C., 23° C., 24° C., 25° C., 26° C., 27° C., 28° C., 29° C., or 30° C.

According to aspects of the present disclosure, the test sample is stored at the first storage temperature for a first period of time typically in the range of 1 hour to 2 years, but may be longer or shorter, prior to extracting the analyte from the test sample to produce an extracted sample.

According to aspects of the present disclosure, the test sample is stored at the second storage temperature for a second period of time typically in the range of 1 hour to 2 years, but may be longer or shorter, following the first period of time and prior to extracting the analyte from the test sample to produce an extracted sample.

According to aspects of the present disclosure, extracting the analyte from the test sample to produce an extracted sample is performed within one hour after drying the biological sample on the treated support.

Extraction of the analyte from the test sample according to aspects of the present disclosure includes contacting the test sample with one or more liquid solvents such that the analyte is thereby removed from the solid support and transferred into the one or more liquid solvents. Extraction of the analyte from the test sample according to aspects of the present disclosure includes placing the test sample, or a portion thereof, in a container with the one or more liquid solvents.

As a non-limiting example, the treated support is a substantially planar filter paper and the biological sample is applied, producing a test sample. The test sample is placed in a container, such as a cup, microfuge tube, or vial. One or more solvents are present in the container such that the test sample is in contact with the one or more solvents. Optionally, the test sample is subjected to agitation, such as by inversion, stirring, vortexing or a combination of any of these, to stimulate release of the analyte from the test sample into the one or more solvents.

A solvent used to extract an analyte from a test sample is compatible with the analyte such that the analyte is not degraded by the solvent. Solvents used to extract an analyte from a test sample include, but are not limited to, water, aqueous buffers, water-miscible organic solvents, and mixtures of any two or more thereof.

Aqueous buffers useful in extraction of the analyte from the test sample include, but are not limited to, barbital buffer, bicarbonate buffer, bicine buffer, N,N′-bis(2-hydroxyethyl)glycine (BIC IN), 2-[bis(2-hydroxyethyl)amino]-2-(hydroxymethyl)-1,3-propanediol (BISTRIS), borate buffer, cacodylate buffer, 2-(cyclohexylamino)ethane-2-sulfonic acid (CHES), citrate buffer, glycine buffer, N-2-(hydroxyethyl)piperazine-N′-2-ethanesulfonic acid (HEPES) buffer, N-(2-hydroxyethyl)piperazine-N′-3-propanesulfonic acid (HEPPS) buffer, 2-(N-morpholino)ethanesulfonic acid (MES) buffer, 3-(N-morpholino)propanesulfonic acid (MOPS) buffer, phosphate buffer, piperazine-N,N′-bis(2-ethanesulfonic acid (PIPES) buffer, [tris(hydroxymethyl)methylamino]propanesulfonic acid (TAPS) buffer, 2-[Tris(hydroxymethyl)-methylamino]-ethanesulfonic acid (TES) buffer, N-tris(hydroxymethyl)methylglycine (tricine) buffer, triethanolamine buffer, and tris(hydroxymethyl)aminomethane (Tris) buffer. Aqueous buffers are typically used at concentrations in the range of 1 to 500 mM, such as 5 to 100 mM, or such as 10 to 50 mM, and have a pH in the range of pH 4 to pH 9.

Water-miscible organic solvents include, but are not limited to, alcohols, acetone, acetonitrile, dioxane, tetrahydrofuran, acetaldehyde, acetic acid, butyric acid, diethanolamine, diethylenetriamine, dimethylformamide, dimethoxyethane, dimethyl sulfoxide, ethylamine, ethylene glycol, formic acid, glycerol, methyl diethanolamine, methyl isocyanide, N-Methyl-2-pyrrolidone, propanoic acid, propylene glycol, pyridine, triethylene glycol, and any two or more thereof.

Alcohols useful in extraction of the analyte from the test sample include, but are not limited to, methanol, ethanol, 1-propanol, 2-propanol, 1,2-butanediol, 1,3-butanediol, 1,4-butanediol, 2-butoxyethanol, furfuryl alcohol, 1,3-propanediol, 1,5-pentanediol, and any two or more thereof.

According to aspects of the present disclosure, mass spectrometry is used in a method for assessing one more analytes in an extracted sample.

A variety of configurations of mass spectrometers can be used in a method of the present disclosure. In general, a mass spectrometer has the following major components: a sample inlet, an ion source, a mass analyzer, a detector, a vacuum system, and instrument-control system, and a data system. Common mass analyzers include a quadrupole mass filter, ion trap mass analyzer and time-of-flight mass analyzer. One such example is the PerkinElmer® QSight® MD screening system.

The ion formation process is a starting point for mass spectrum analysis and several ionization methods are available. For example, electrospray ionization (ESI) can be used. Generally described, in ESI a solution containing the material to be analyzed is passed through a fine needle at high potential which creates a strong electrical field resulting in a fine spray of highly charged droplets that is directed into the mass spectrometer. Other ionization procedures include, for example, fast-atom bombardment (FAB) which uses a high-energy beam of neutral atoms to strike a solid sample causing desorption and ionization. Matrix-assisted laser desorption ionization (MALDI) is a method in which a laser pulse is used to strike a sample that has been crystallized in an UV-absorbing compound matrix. Other ionization procedures known in the art include, for example, plasma and glow discharge, plasma desorption ionization, resonance ionization, and secondary ionization.

Electrospray ionization (ESI) has several properties that are useful for methods of assessing an analyte of the present disclosure. For example, the efficiency of ESI can be very high which provides the basis for highly sensitive measurements. Furthermore, ESI produces charged molecules from solution, which is convenient for analyzing analytes and standards that are in solution. In contrast, ionization procedures such as MALDI require crystallization of the material to be analyzed prior to ionization.

Since ESI can produce charged molecules directly from solution, it is compatible with samples from liquid chromatography systems. In liquid chromatography with tandem mass spectrometry (LC-MS-MS), the inlet can be a capillary-column liquid chromatography source. For example, a mass spectrometer can have an inlet for a liquid chromatography system, such as an HPLC, so that fractions flow from the chromatography column into the mass spectrometer. This in-line arrangement of a liquid chromatography system and mass spectrometer is sometimes referred to as LC-MS. An LC-MS system can be used, for example, to separate analytes and standards from complex mixtures before mass spectrometry analysis. In addition, chromatography can be used to remove salts or other buffer components from the sample before mass spectrometry analysis. For example, desalting of a sample using a reversed-phase HPLC column, in-line or off-line, can be used to increase the efficiency of the ionization process and thus improve sensitivity of detection by mass spectrometry.

A variety of mass analyzers are available that can be paired with different ion sources. Different mass analyzers have different advantages as known to one skilled in the art and as described herein. The mass spectrometer and methods chosen for detection depends on the particular assay, for example, a more sensitive mass analyzer can be used when a small amount of ions are generated for detection. Several types of mass analyzers and mass spectrometry methods are described below.

Quadrupole mass spectrometry utilizes a quadrupole mass filter or analyzer. This type of mass analyzer is composed of four rods arranged as two sets of two electrically connected rods. A combination of rf and dc voltages are applied to each pair of rods which produces fields that cause an oscillating movement of the ions as they move from the beginning of the mass filter to the end. The result of these fields is the production of a high-pass mass filter in one pair of rods and a low-pass filter in the other pair of rods. Overlap between the high-pass and low-pass filter leaves a defined m/z that can pass both filters and traverse the length of the quadrupole. This m/z is selected and remains stable in the quadrupole mass filter while all other m/z have unstable trajectories and do not remain in the mass filter. A mass spectrum results by ramping the applied fields such that an increasing m/z is selected to pass through the mass filter and reach the detector. In addition, quadrupoles can also be set up to contain and transmit ions of all m/z by applying an rf-only field. This allows quadrupoles to function as a lens or focusing system in regions of the mass spectrometer where ion transmission is needed without mass filtering. This will be of use in tandem mass spectrometry as described further below.

A quadrupole mass analyzer, as well as the other mass analyzers described herein, can be programmed to analyze a defined m/z or mass range. Since the mass range of analytes and standards will be known prior to an assay, a mass spectrometer can be programmed to transmit ions of the projected correct mass range while excluding ions of a higher or lower mass range. The ability to select a mass range can decrease the background noise in the assay and thus increase the signal-to-noise ratio as well as increasing the specificity of the assay. Therefore, the mass spectrometer can accomplish an inherent separation step as well as detection and identification of analytes and standards.

Ion trap mass spectrometry utilizes an ion trap mass analyzer. In these mass analyzers, fields are applied so that ions of all m/z are initially trapped and oscillate in the mass analyzer. Ions enter the ion trap from the ion source through a focusing device such as an octapole lens system. Ion trapping takes place in the trapping region before excitation and ejection through an electrode to the detector. Mass analysis is accomplished by sequentially applying voltages that increase the amplitude of the oscillations in a way that ejects ions of increasing m/z out of the trap and into the detector. In contrast to quadrupole mass spectrometry, all ions are retained in the fields of the mass analyzer except those with the selected m/z. One advantage to ion traps is that they have very high sensitivity, as long as one is careful to limit the number of ions being tapped at one time. Control of the number of ions can be accomplished by varying the time over which ions are injected into the trap. The mass resolution of ion traps is similar to that of quadrupole mass filters, although ion traps do have low m/z limitations.

Time-of-flight mass spectrometry utilizes a time-of-flight mass analyzer. For this method of m/z analysis, an ion is first given a fixed amount of kinetic energy by acceleration in an electric field (generated by high voltage). Following acceleration, the ion enters a field-free or “drift” region where it travels at a velocity that is inversely proportional to its m/z. Therefore, ions with low m/z travel more rapidly than ions with high m/z. The time required for ions to travel the length of the field-free region is measured and used to calculate the m/z of the ion. One consideration in this type of mass analysis is that the set of ions being studied be introduced into the analyzer at the same time. For example, this type of mass analysis is well suited to ionization techniques like MALDI which produces ions in short well-defined pulses. Another consideration is to control velocity spread produced by ions that have variations in their amounts of kinetic energy. The use of longer flight tubes, ion reflectors, or higher accelerating voltages can help minimize the effects of velocity spread. Time-of-flight mass analyzers have a high level of sensitivity and a wider m/z range than quadrupole or ion trap mass analyzers. Also data can be acquired quickly with this type of mass analyzer because no scanning of the mass analyzer is necessary.

Tandem mass spectrometry can utilize combinations of the mass analyzers described above.

Tandem mass spectrometers can use a first mass analyzer to separate ions according to their m/z in order to isolate an ion of interest for further analysis. The isolated ion of interest is then broken into fragment ions, called collisionally activated dissociation or collisionally induced dissociation, and the fragment ions are analyzed by the second mass analyzer. These types of tandem mass spectrometer systems are called tandem in space systems because the two mass analyzers are separated in space, usually by a collision cell. Tandem mass spectrometer systems also include tandem in time systems where one mass analyzer is used, however the mass analyzer is used sequentially to isolate an ion, induce fragmentation, and then perform mass analysis.

Mass spectrometers in the tandem in space category have more than one mass analyzer. For example, a tandem quadrupole mass spectrometer system can have a first quadrupole mass filter, followed by a collision cell, followed by a second quadrupole mass filter and then the detector. Another arrangement is to use a quadrupole mass filter for the first mass analyzer and a time-of-flight mass analyzer for the second mass analyzer with a collision cell separating the two mass analyzers.

Other tandem systems are known in the art including reflection-time-of-flight, tandem sector and sector-quadrupole mass spectrometry.

Mass spectrometers in the tandem in time category have one mass analyzer that performs different functions at different times. For example, an ion trap mass spectrometer can be used to trap ions of all m/z. A series of rf scan functions are applied which ejects ions of all m/z from the trap except the m/z of ions of interest. After the m/z of interest has been isolated, an rf pulse is applied to produce collisions with gas molecules in the trap to induce fragmentation of the ions. Then the m/z values of the fragmented ions are measured by the mass analyzer. Ion cyclotron resonance instruments, also known as Fourier transform mass spectrometers, are an example of tandem-in-time systems.

Several types of tandem mass spectrometry experiments can be performed by controlling the ions that are selected in each stage of the experiment. The different types of experiments utilize different modes of operation, sometimes called “scans,” of the mass analyzers. In a first example, called a mass spectrum scan, the first mass analyzer and the collision cell transmit all ions for mass analysis into the second mass analyzer. In a second example, called a product ion scan, the ions of interest are mass-selected in the first mass analyzer and then fragmented in the collision cell. The ions formed are then mass analyzed by scanning the second mass analyzer. In a third example, called a precursor ion scan, the first mass analyzer is scanned to sequentially transmit the mass analyzed ions into the collision cell for fragmentation. The second mass analyzer mass-selects the product ion of interest for transmission to the detector. Therefore, the detector signal is the result of all precursor ions that can be fragmented into a common product ion. Other experimental formats include neutral loss scans where a constant mass difference is accounted for in the mass scans. The use of these different tandem mass spectrometry scan procedures can be advantageous when large sets of analytes are measured in a single experiment.

In view of the above, those skilled in the art recognize that different mass spectrometry methods, for example, quadrupole mass spectrometry, ion trap mass spectrometry, time-of-flight mass spectrometry and tandem mass spectrometry, can use various combinations of ion sources and mass analyzers which allows for flexibility in designing customized detection protocols. In addition, mass spectrometers can be programmed to transmit all ions from the ion source into the mass spectrometer either sequentially or at the same time. Furthermore, a mass spectrometer can be programmed to select ions of a particular mass for transmission into the mass spectrometer while blocking other ions. The ability to precisely control the movement of ions in a mass spectrometer allows for greater options in detection protocols which can be advantageous when a large number of analytes are being analyzed.

Different mass spectrometers have different levels of resolution, that is, the ability to resolve peaks between ions closely related in mass. The resolution is defined as R=m/delta m, where m is the ion mass and delta m is the difference in mass between two peaks in a mass spectrum. For example, a mass spectrometer with a resolution of 1000 can resolve an ion with a m/z of 100.0 from an ion with a m/z of 100.1. Those skilled in the art will therefore select a mass spectrometer having a resolution appropriate for the analyte(s) to be detected.

Mass spectrometers can resolve ions with small mass differences and measure the mass of ions with a high degree of accuracy. Therefore, analytes of similar masses can be used together in the same experiment since the mass spectrometer can differentiate the mass of even closely related molecules. The high degree of resolution and mass accuracy achieved using mass spectrometry methods allows the use of large sets of analytes because they can be distinguished from each other.

Mass spectrometry devices and general methods of their use are well known in the art as exemplified in McMaster, M., LC/MS A Practical User's Guide, 2005, John Wiley & Sons, USA; and Hoffmann and Stroobant, Mass Spectrometry Principles and Applications, 2007, John Wiley & Sons, England.

According to aspects of the present disclosure, a standard is used in a method of assessing an analyte. Standards suitable for assays are well-known in the art and the standard used can be any appropriate standard.

In one example, a standard is a result of an assay of the one or more analytes to be assessed in a comparable biological sample from a control subject.

A standard may be a reference level of the one or more analytes previously determined in a biological sample and stored in a print or electronic medium for recall and comparison to a result produced according to a method of assessing the one or more analytes by a method of the present disclosure.

A standard can be a result of an assay of the one or more analytes in a comparable biological sample from a subject at a different time. For example, a standard can be a result of an assay of the one or more analytes in a comparable biological sample obtained from the same subject at a different time.

A standard can be an average level of one or more analytes in comparable samples obtained from one or more populations of subjects. The “average level” is determined by assay of the one or more analytes in comparable samples obtained from each individual subject of the population. The term “comparable sample” is used to indicate that the samples are of the same type, i.e. each of the comparable samples is a blood sample, for example.

According to aspects of the present disclosure, a standard is added to the test sample.

According to aspects of the present disclosure, one or more standards can be added to a test sample prior to analysis, such as by LC-MS-MS.

Such standards can be useful when the methods are carried out in a quantitative format, for example.

According to aspects of the present disclosure, a method assessing an analyte can be used quantitatively, if desired, to allow comparison of results with known or pre-determined standard amounts of a particular analyte.

A method assessing an analyte according to the present disclosure can be used qualitatively when the presence of the analyte in the sample is indicative of a health status of a subject, for example, when the analyte assessed is the result of abnormal metabolic processes, and/or is not detected in a normal sample. The methods can also be used qualitatively when a biological sample is compared with a reference sample, which can be either a normal reference or a disorder reference. In this format, the relative amount of analyte can be indicative of a disorder.

According to aspects of the present disclosure, an internal standard for an analyte useful in a method of the disclosure can be any modification or analog of the analyte that is detectable by mass spectrometry. Such a standard is separately detectable from the analyte to be assessed based on a unique physical characteristic, such as a unique mass or charge-to-mass ratio. Alternatively, or in addition, a suitable generic reference standard can be used. Such an internal standard will, for example, co-elute with the analyte if a separation method such as chromatography is used prior to mass spectrometric analysis, such as in LC-MS-MS. A commonly used internal standard for mass spectrometry is a stable isotopically labeled form or chemical derivative of the analyte, such as a deuterated form of the analyte.

The reference level can be determined by a plurality of methods, provided that the resulting reference level accurately provides an amount of metabolic analyte or enzyme activity above which exists a first group of individuals having a different probability of metabolic disorder than that of a second group of individuals having metabolic analyte or enzyme activity amount below the reference level. The reference level can be determined by comparison of metabolic analyte or enzyme activity amount in populations of patients having the same metabolic disorder. This can be accomplished, for example, by histogram analysis, in which an entire cohort of patients are graphically presented, wherein a first axis represents the amount of metabolic analyte or enzyme activity and a second axis represents the number of individuals in the cohort whose biological sample contains an analyte at a given amount. Two or more separate groups of individuals can be determined by identification of subsets populations of the cohort which have the same or similar levels of an analyte. Determination of the reference level can then be made based on an amount which best distinguishes these separate groups. The reference level can be a single number, equally applicable to every individual, or the reference level can vary, according to specific subpopulations of individuals. For example, older individuals might have a different reference level than younger individuals for the same analyte.

A difference detected in levels or expression of one or more analytes in assays of the present disclosure compared to a standard can be an increase or decrease in level or expression of the one or more analytes. The magnitude of the increase or decrease can be, for example, at least 5%, 10%, 15%, 20%, 25%, 30%, 35%, 40%, 45%, 50%, 55%, 60%, 65%, 70%, 75%, 80%, 85%, 90%, 95%, 99%, or more, of the standard level.

Assay results can be analyzed using statistical analysis by any of various methods, exemplified by parametric or non-parametric tests, analysis of variance, analysis of covariance, logistic regression for multivariate analysis, Fisher's exact test, the chi-square test, Student's T-test, the Mann-Whitney test, Wilcoxon signed ranks test, McNemar test, Friedman test and Page's L trend test. These and other statistical tests are well-known in the art as detailed in Hicks, C M, Research Methods for Clinical Therapists: Applied Project Design and Analysis, Churchill Livingstone (publisher); 5th Ed., 2009; and Freund, R J et al., Statistical Methods, Academic Press; 3rd Ed., 2010.

Methods of assessing an analyte according to aspects of the present invention surprisingly demonstrate a lower limit of detection (LOD) and/or lower limit of quantitation (LOQ) compared to prior methods.

The term “‘limit of detection” or “LOD” refers to the lowest concentration of an analyte that an assessment method can reliably differentiate from background noise.

The term “limit of quantitation” or “LOQ”, also known as “limit of quantification”, refers to the lowest amount of an analyte in a sample that can be quantitatively determined with acceptable precision and accuracy, with a relative standard deviation (RSD %) of 30% and an accuracy of 70% to 130%.

According to aspects of the present disclosure, NAD is surprisingly detectable at a concentration of 5 μM or higher in the biological sample.

Embodiments of inventive compositions and methods are illustrated in the following examples. These examples are provided for illustrative purposes and are not considered limitations on the scope of inventive compositions and methods.

### EXAMPLES

### Example 1

A surrogate matrix consisting of a 1:1 dilution of human packed red blood cells and 2% human serum albumin was spiked with three levels of NAD: 7.5, 67.8 μM, and 452 μM.

The spiked surrogate matrix was spotted onto untreated cotton linter filter paper supports. After application of the samples, the supports were processed for assessment of NAD after drying and after storage at 15-30° C. for 3 days.

For each condition, a 3.2 mm punch was removed from the respective support at the location of the blood sample on the support and these were placed in separate wells of a microplate. 150 μL of an 80% methanol extraction solution containing 1.5 μM of NAD-d₅ (internal standard) was added to each punch in the respective microfuge tube. The plate was then sealed and centrifuged for 1 minute at 2500 rpm, followed by incubation for 30 min at 25° C. with 800 rpm agitation. The plate was then centrifuged for 1 minute at 2500 rpm. The extraction solution containing the extracted sample was transferred and dried down using nitrogen gas. The residue was reconstituted with 100 μL of 70% acetonitrile and then mixed for 10 min at 25° C. with 400 rpm agitation. The extracted samples were analyzed by LC MS/MS. NAD concentration was calculated using a calibration curve.

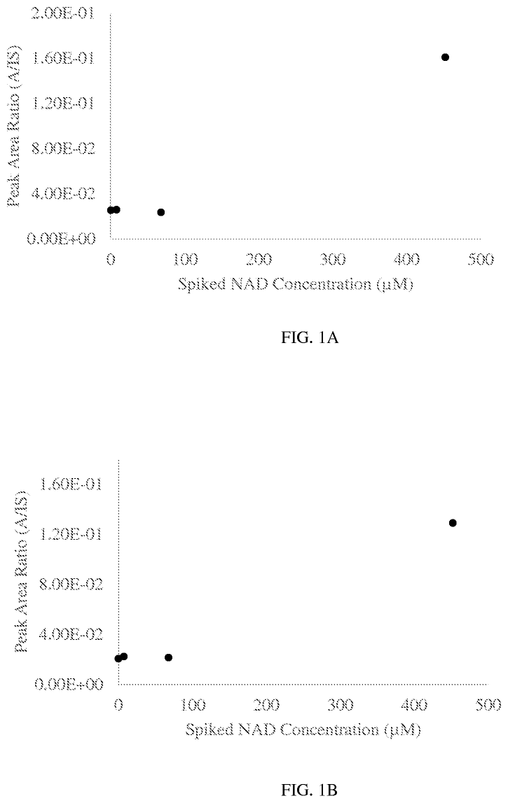

FIG. 1A shows that NAD was not detected within the endogenous (no NAD added), 5 or 45 μg/mL spikes. It was only detected in the very high concentration spike (300 μg/mL).

FIG. 1B shows that similar results were obtained after 3 days of storage. This study indicated that loss of NAD occurred while the blood was drying on the untreated support. Once the samples on the supports were dried, additional loss was minimal.

### Example 2

A surrogate matrix consisting of a 1:1 dilution of human packed red blood cells and 2% human serum albumin was spiked with three levels of NAD: 7.5, 67.8 μM, and 452 μM.

The spiked surrogate matrix was spotted onto filter paper supports coated with a composition including ethylenediaminetetraacetic acid (EDTA), tris(hydroxymethyl)aminomethane (Tris), sodium dodecyl sulfate (SDS), and uric acid. After application of the samples, the supports were processed for assessment of NAD after drying and after storage at 15-30° C. for 3 days.

For each condition, a 3.2 mm punch was removed from the respective support at the location of the blood sample on the support and these were placed in separate wells in a microplate. 150 μL of an 80% methanol extraction solution containing 1.5 μM of NAD-d₅ (internal standard) was added to each punch in the respective well. The plate was then sealed and centrifuged for 1 minute at 2500 rpm, followed by incubation for 30 min at 25° C. with 800 rpm agitation. The plate was then centrifuged for 1 minute at 2500 rpm. The extraction solution containing the extracted sample was transferred and dried down using nitrogen gas. The residue was reconstituted with 100 μL of 70% acetonitrile and then mixed for 10 min at 25° C. with 400 rpm agitation. The extracted samples were analyzed by LC MS/MS. NAD concentration was calculated using a calibration curve.

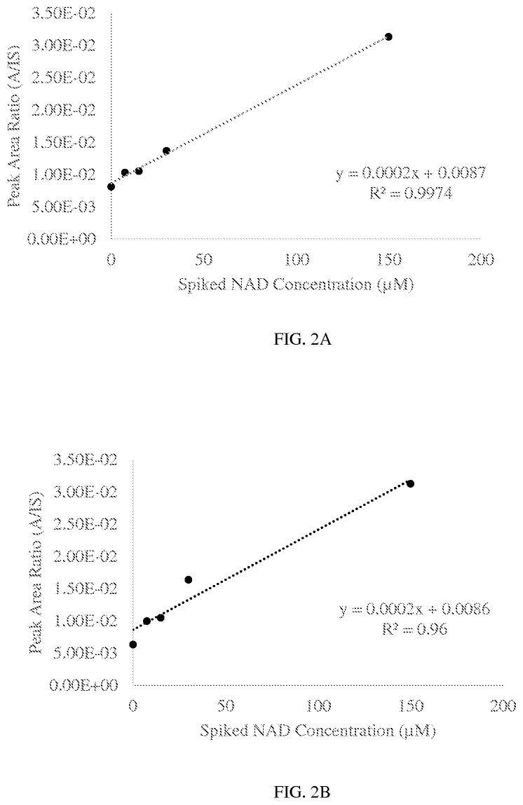

FIGS. 2A and 2B: Response (peak area ratio) vs. spiked NAD concentration plots from analysis of surrogate matrix spotted onto Whatman® FTA cards. (A) analysis immediately after drying time, (B) analysis after storage at 15-30° C. for 3 days.

FIGS. 2A and 2B demonstrate that a linear relationship was observed and that NAD was detected in all spiking levels.

FIG. 2B further shows that minimal loss of NAD was observed after 3 days of storage at 15-30° C.

### Example 3

In order to investigate the extent of inhibition of enzyme activity, blood samples were collected from five individual humans and aliquots from each sample were spotted onto untreated filter paper supports or filter paper supports coated with ethylenediaminetetraacetic acid (EDTA), tris(hydroxymethyl)aminomethane (Tris), sodium dodecyl sulfate (SDS), and uric acid. The supports were processed after drying and after storage at 15-30° C. for 3 days.

The activity of 13 different lysosomal enzymes were measured from each card type. Table 1 below provides the percent reduction of activity for each enzyme. These results are the average of the five individuals.

TABLE 1

| | Average loss of enzyme | | Average loss of enzyme |
|---|---|---|---|
| Enzyme | activity (%) | Enzyme | activity (%) |
| ABG | 28.8 | bGAL | 70.5 |
| ASM | 9.7 | GALNS | 0 |
| GAA | 29.1 | GUSB | 25.2 |
| GALC | 70.5 | ID2S | 0 |
| GLA | 87.5 | NAGLU | 0 |
| IDUA | 34.1 | TPP1 | 11.0 |
| ARSB | 0 | | |

Table 1 shows that three of the enzymes assayed had a >70% reduction in enzyme activity, four of the enzymes assayed had a 25-70% reduction, two of the enzymes assayed a 5-10% reduction, and four of the enzymes assayed were not inhibited.

### Example 4

Blood is collected from a human subject using a finger prick or heel stick and deposited on a filter paper support which is either, untreated, or which is treated with the indicated protein denaturant. The blood is allowed to dry on the support for approximately 3 hours. Two, 3.2 mm punches are removed from the support at the location of the blood sample on the support and these are placed in separate wells in a microplate. 150 μL of an 80% methanol extraction solution containing 1.5 μM of NAD-d₅ (internal standard) is added to each punch in the microplate. The plate is then sealed and centrifuged for 1 minute at 2500 rpm, followed by incubation for 30 min at 25° C. with 800 rpm agitation. The plate is then centrifuged for 1 minute at 2500 rpm. The extraction solution containing the extracted sample is transferred and dried down using nitrogen gas. The residue is reconstituted with 100 μL of 70% acetonitrile and then mixed for 10 min at 25° C. with 400 rpm agitation. The extracted samples are analyzed by LC MS/MS. NAD concentration is calculated using a calibration curve.

### Example 5

In order to increase the extent of inhibition of enzyme activity, filter paper supports were pre-treated with a protein denaturing agent in five increasing concentrations (Treated Supports #2, #3, #4, #5, #6) for comparison with a composition (Treated Support #1) including ethylenediaminetetraacetic acid (EDTA), tris(hydroxymethyl)aminomethane (Tris), sodium dodecyl sulfate (SDS), and uric acid. A blood sample was collected from one human and aliquots from the sample were spotted onto untreated filter paper supports pre-treated with the five concentrations of the protein denaturing agent (Treated Supports #2, #3, #4, #5, #6) or onto filter paper supports pre-treated with the composition (Treated Support #1). After application of the samples, the supports were assayed for enzyme activity after drying.

The activity of 13 different lysosomal enzymes were measured from each support type. Table 2 provides the percent reduction of activity for each enzyme

TABLE 2

| | Average loss of enzyme activity (%) | | | | | |
|---|---|---|---|---|---|---|
| | Treated | Treated | Treated | Treated | Treated | (Reference) |
| | Support | Support | Support | Support | Support | Treated |
| Enzyme | #2 | #3 | #4 | #5 | #6 | Support #1 |
| ABG | 30.8 | 99.3 | 99.8 | 100.4 | 100.4 | 29.0 |
| ASM | 51.3 | 99.5 | 100.0 | 99.8 | 100.0 | 0 |
| GAA | 54.8 | 98.7 | 100.0 | 100.0 | 100.0 | 27.7 |
| GALC | 23.8 | 92.9 | 99.4 | 100.0 | 100.3 | 63.2 |
| GLA | 71.4 | 99.4 | 100.0 | 99.8 | 99.7 | 92.9 |
| IDUA | 32.9 | 99.3 | 100.2 | 100.2 | 100.4 | 0 |
| ARSB | 0 | 0 | 98.8 | 96.1 | 100.4 | 0 |
| bGAL | 0 | 0 | 100.0 | 100.0 | 99.9 | 54.2 |
| GALNS | 0 | 0 | 100.4 | 100.1 | 100.4 | 0 |
| GUSB | 11.5 | 39.0 | 100.1 | 100.1 | 100.4 | 9.0 |
| ID2S | 70.6 | 98.8 | 99.6 | 100.3 | 100.2 | 0 |
| NAGLU | 50.2 | 89.4 | 100.6 | 100.5 | 100.0 | 0 |
| TPPI | 11.1 | 35.9 | 100.0 | 100.0 | 100.0 | 18.1 |

### Example 6

Filter paper supports were pre-treated with a protein denaturing agent in five increasing concentrations (Treated Supports #2, #3, #4, #5, #6) for comparison with an untreated filter paper support and a filter paper support treated with a composition including ethylenediaminetetraacetic acid (EDTA), tris(hydroxymethyl)aminomethane (Tris), sodium dodecyl sulfate (SDS), and uric acid. A blood sample was collected from one human and aliquots from the sample were spotted onto filter paper supports pre-treated with the five concentrations of the protein denaturing agent, the untreated filter paper support, or onto filter paper supports pre-treated with the composition. After application of the samples, the supports were processed for assessment of NAD after drying.

For each condition, a 3.2 mm punch was removed from the respective support at the location of the blood sample on the support and these were placed in separate wells in a microplate. 150 μL of an 80% methanol extraction solution containing 1.5 μM f NAD-d₅ (internal standard) was added to each punch in the respective well. The plate was then sealed and centrifuged for 1 minute at 2500 rpm, followed by incubation for 30 min at 25° C. with 800 rpm agitation. The plate was then centrifuged for 1 minute at 2500 rpm. The extraction solution containing the extracted sample was transferred and dried down using nitrogen gas. The residue was reconstituted with 100 μL of 70% acetonitrile and then mixed for 10 min at 25° C. with 400 rpm agitation. The extracted samples were analyzed by LC MS/MS. NAD concentration was calculated using a calibration curve.

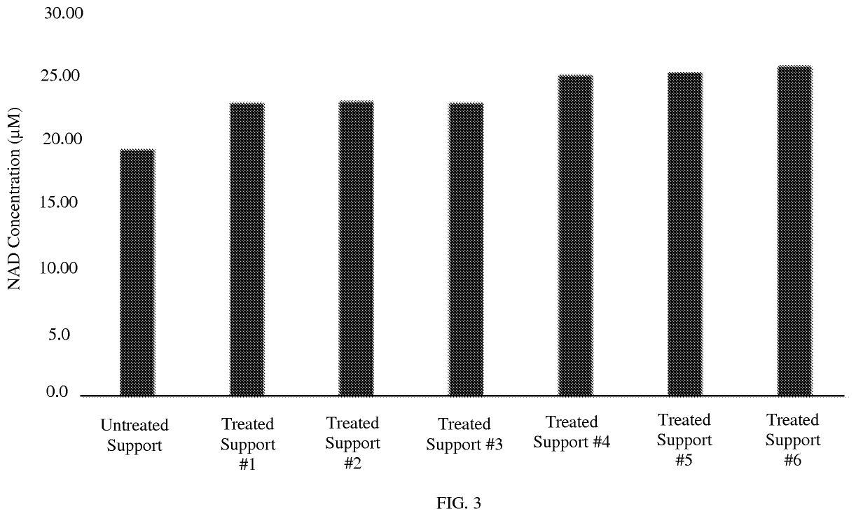

FIG. 3 shows results of assessing NAD concentrations measured from seven different supports, including 6 treated supports (Treated Supports #1, #2, #3, #4, #5, #6) and 1 untreated support. Treated Supports #2, #3, #4, #5, #6 have increasing amounts of a protein denaturant.

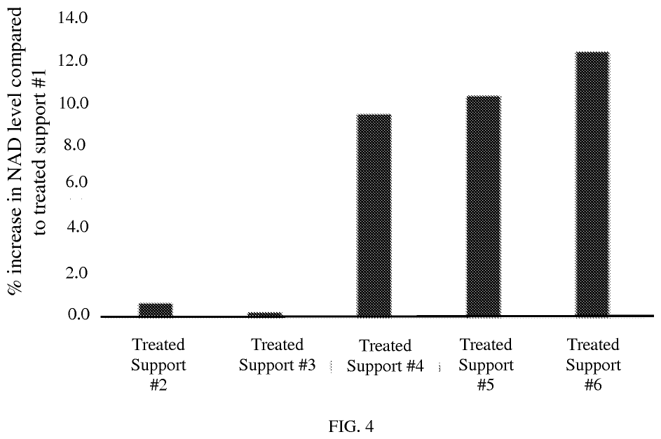

FIG. 4. Percentage increase in measured NAD concentration in comparison to Treated Support #1.

Items

Item 1. A method of assessing nicotinamide adenine dinucleotide (NAD) in a blood sample, comprising: applying a protein denaturant to a support, producing a treated support; applying a blood sample to the treated support, the blood sample comprising or suspected of comprising NAD, wherein the NAD is a substrate for an enzyme present, or suspected of being present, in the blood sample, wherein the protein denaturant inhibits enzymatic activity of the enzyme on NAD; drying the blood sample on the treated support, producing a test sample; extracting NAD from the test sample, producing an extracted sample; and subjecting the extracted sample to liquid chromatography tandem mass spectrometry (LC/MS/MS), thereby assessing NAD in the blood sample.

Item 2. The method of item 1, wherein the test sample is stored a first storage temperature in the range of −70° C. to 40° C. for a first period of time following the drying and prior to the extracting.

Item 3. The method of item 2, wherein the first storage temperature is in the range of −20° C. to 4° C.

Item 4. The method of item 2, wherein the first storage temperature is in the range of −15° C. to 30° C.

Item 5. The method of any of items 2 to 4, wherein the first period of time is in the range of 1 hour to 2 years.

Item 6. The method of any of items 2 to 5, further comprising storing the test sample at a second storage temperature in the range of −70° C. to 4° C. for a second period of time following the first period of time and prior to the extracting.

Item 7. The method of item 6, wherein the second period of time is in the range of 1 hour to 2 years.

Item 8. The method of item 1, wherein the extracting is performed within one hour after the drying.

Item 9. The method of any of items 1 to 8, wherein a reference is added to the blood sample, the test sample, or both the blood sample and the test sample.

Item 10. The method of any of items 1 to 9, wherein the support comprises filter paper.

Item 11. The method of any of items 1 to 10, wherein the support is substantially planar.

Item 12. The method of any of items 1 to 11, wherein the blood sample is obtained from a subject.

Item 13. The method of item 12, wherein the subject is human.

Item 14. The method of any of items 1 to 13, wherein NAD is detectable at a concentration of 5 μM or higher in the blood sample.

Item 15. A method of assessing an analyte in a biological sample, comprising: applying a protein denaturant to a support, producing a treated support; applying a biological sample to the treated support, the biological sample comprising or suspected of comprising an analyte, wherein the analyte is a substrate for an enzyme present, or suspected of being present, in the biological sample, wherein the protein denaturant inhibits enzymatic activity of the enzyme on the analyte; drying the biological sample on the treated support, producing a test sample; extracting the analyte from the treated support, producing an extracted sample; and subjecting the extracted sample to liquid chromatography tandem mass spectrometry (LC/MS/MS), thereby assessing the analyte in the biological sample.

Item 16. The method of item 15, wherein the test sample is stored a first storage temperature in the range of −70° C. to 40° C. for a first period of time following the drying and prior to the extracting.

Item 17. The method of item 16, wherein the first storage temperature is in the range of −20° C. to 4° C.

Item 18. The method of item 16, wherein the first storage temperature is in the range of −15° C. to 30° C.

Item 19. The method of any of items 15 to 18, wherein the first period of time is in the range of 1 hour to 2 years.

Item 20. The method of any of items 15 to 19, further comprising storing the test sample at a second storage temperature in the range of −70° C. to 4° C. for a second period of time following the first period of time and prior to the extracting.

Item 21. The method of item 20, wherein the second period of time is in the range of 1 hour to 2 years.

Item 22. The method of item 15, wherein the extracting is performed within one hour after the drying.

Item 23. The method of any of items 15 to 22, wherein a reference is added to the blood sample, the test sample, or both the blood sample and the test sample.

Item 24. The method of any of items 15 to 23, wherein the support comprises filter paper.

Item 25. The method of any of items 15 to 24, wherein the support is substantially planar.

Item 26. The method of any of items 15 to 25, wherein the biological sample is obtained from a subject.

Item 27. The method of item 26, wherein the subject is human.

Item 28. A method of assessing an analyte in a biological sample, comprising: extracting the analyte from a biological sample dried on a treated support, producing an extracted sample, the treated support comprising a protein denaturant, wherein the analyte is a substrate for an enzyme present, or suspected of being present, in the biological sample, wherein the protein denaturant inhibits enzymatic activity of the enzyme on the analyte; and subjecting the extracted sample to liquid chromatography tandem mass spectrometry (LC/MS/MS), thereby assessing the analyte in the biological sample.

Item 29. The method of item 28, wherein the test sample is stored a first storage temperature in the range of −70° C. to 40° C. for a first period of time following the drying and prior to the extracting.

Item 30. The method of item 29, wherein the first storage temperature is in the range of −20° C. to 4° C.

Item 31. The method of item 29, wherein the first storage temperature is in the range of −15° C. to 30° C.

Item 32. The method of any of items 29 to 31, wherein the first period of time is in the range of 1 hour to 2 years.

Item 33. The method of any of items 29 to 32, further comprising storing the test sample at a second storage temperature in the range of −70° C. to 4° C. for a second period of time following the first period of time and prior to the extracting.

Item 34. The method of item 33, wherein the second period of time is in the range of 1 hour to 2 years.

Item 35. The method of item 28, wherein the extracting is performed within one hour after the biological sample is dried on the treated support.

Item 36. The method of any of items 28 to 35, wherein a reference is added to the biological sample dried on a treated support, the extracted sample, or both the biological sample dried on a treated support and the extracted sample.

Item 37. The method of any of items 28 to 36, wherein the support comprises filter paper.

Item 38. The method of any of items 28 to 37, wherein the support is substantially planar.

Item 39. The method of any of items 28 to 38, wherein the biological sample is a biological sample of a subject.

Item 40. The method of item 39, wherein the subject is human.

Item 41. A method of assessing NAD in a biological sample substantially as shown and/or described.

Item 42. A method of assessing an analyte in a biological sample substantially as shown and/or described.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of assessing nicotinamide adenine dinucleotide (NAD) in a blood sample, comprising:
applying a protein denaturant to a support, producing a treated support;
applying a blood sample to the treated support, the blood sample comprising or suspected of comprising NAD, wherein the NAD is a substrate for an enzyme present, or suspected of being present, in the blood sample, wherein the protein denaturant inhibits enzymatic activity of the enzyme on NAD;
drying the blood sample on the treated support, producing a test sample;
extracting NAD from the test sample, producing an extracted sample; and
subjecting the extracted sample to liquid chromatography tandem mass spectrometry (LC/MS/MS), thereby assessing NAD in the blood sample.

2. The method of claim 1, wherein the test sample is stored a first storage temperature in the range of −70° C. to 40° C. for a first period of time following the drying and prior to the extracting.

3. The method of claim 2, wherein the first storage temperature is in the range of −20° C. to 4° C.

4. The method of claim 2, wherein the first storage temperature is in the range of −15° C. to 30° C.

5. The method of claim 2, wherein the first period of time is in the range of 1 hour to 2 years.

6. The method of claim 2, further comprising storing the test sample at a second storage temperature in the range of −70° C. to 4° C. for a second period of time following the first period of time and prior to the extracting, wherein the first storage temperature is different than the second storage temperature.

7. The method of claim 6, wherein the second period of time is in the range of 1 hour to 2 years.

8. The method of claim 1, wherein the extracting is performed within one hour after the drying.

9. The method of claim 1, wherein a reference is added to the blood sample, the test sample, or both the blood sample and the test sample.

10. The method of claim 1, wherein the support comprises filter paper.

11. The method of claim 1, wherein the support is substantially planar.

12. The method of claim 1, wherein the blood sample is obtained from a subject.

13. The method of claim 12, wherein the subject is human.

14. The method of claim 1, wherein NAD is detectable at a concentration of 5 μM or higher in the blood sample.

15. A method of assessing an analyte in a biological sample, comprising:
applying a protein denaturant to a support, producing a treated support;
applying a biological sample to the treated support, the biological sample comprising or suspected of comprising an analyte, wherein the analyte is a substrate for an enzyme present, or suspected of being present, in the biological sample, wherein the protein denaturant inhibits enzymatic activity of the enzyme on the analyte;
drying the biological sample on the treated support, producing a test sample;
extracting the analyte from the treated support, producing an extracted sample; and
subjecting the extracted sample to liquid chromatography tandem mass spectrometry (LC/MS/MS), thereby assessing the analyte in the biological sample.

16. The method of claim 15, wherein the test sample is stored a first storage temperature in the range of −70° C. to 40° C. for a first period of time following the drying and prior to the extracting.

17. The method of claim 16, wherein the first storage temperature is in the range of −20° C. to 4° C.

18. The method of claim 16, wherein the first storage temperature is in the range of −15° C. to 30° C.

19. The method of claim 16, wherein the first period of time is in the range of 1 hour to 2 years.

20. The method of claim 16, further comprising storing the test sample at a second storage temperature in the range of −70° C. to 4° C. for a second period of time following the first period of time and prior to the extracting, wherein the first storage temperature is different than the second storage temperature.

21. The method of claim 20, wherein the second period of time is in the range of 1 hour to 2 years.

22. The method of claim 15, wherein the extracting is performed within one hour after the drying.

23. The method of claim 15, wherein a reference is added to the blood sample, the test sample, or both the blood sample and the test sample.

24. The method of claim 15, wherein the support comprises filter paper.

25. The method of claim 15, wherein the support is substantially planar.

26. The method of claim 15, wherein the biological sample is obtained from a subject.

27. The method of claim 26, wherein the subject is human.
